(19) United States
(12) Patent Application Publication
(10) Pub. No.: US 2020/0282107 A1
(43) Pub. Date: Sep. 10, 2020

(54) STERILE CLEAR CONCENTRATED SOLUTION OF BIOCOMPATIBLE COLLAGEN, PROCESS FOR THE PREPARATION AND USE THEREOF

(71) Applicant: IMTEK LTD, Moscow (RU)

(72) Inventors: Egor Olegovich OSIDAK, Moscow (RU); Sergey Petrovich DOMOGATSKY, Moscow (RU); Maria Sergeevna OSIDAK, Moscow (RU)

(73) Assignee: IMTEK LTD, Moscow (RU)

(21) Appl. No.: 16/727,331

(22) Filed: Dec. 26, 2019

(30) Foreign Application Priority Data
Mar. 6, 2019 (RU) 2019106356

(51) Int. Cl. A61L 27/24; A61L 27/52; C07K 1/36; C07K 14/78

(52) U.S. Cl. CPC A61L 27/24; A61L 27/52; B33Y 70/00; C07K 14/78; A61L 2430/40; C07K 1/36

(57) ABSTRACT

The invention relates to the field of medicine and biology. A new product is described that is a sterile clear viscous preparation of a purified collagen dissolved in a 0.1 mM-20 mM aqueous acidic solution, the molecules of said collagen retaining the native triple helix structure in the solution at a temperature of +4° C. to +25° C. A concentrated solution of the collagen forms stable polymeric structures (hydrogels) under physiological conditions and can be used for manufacturing biomedical cell based products and three-dimensional tissue-engineered structures without using chemical and/or photochemical cross-linking. These structures can be heterogeneous density, including structures with a detail resolution of no larger than 0.3 mm, obtained by 3D printing, thus allowing incorporating living cells or cell aggregates used in medical practice in the structure. The disclosed collagen solution will be also useful as a material for forming hydrogels used for cell cultures. The invention also describes a process for the preparation of a sterile clear concentrated solution of biocompatible collagen, and possible uses thereof.

### TECHNICAL FIELD OF THE INVENTION

The invention relates to the field of medicine and biology and provides a new product, specifically a sterile clear viscous aqueous salt solution of a purified collagen preparation dissolved in a 0.1 mM to 20 mM aqueous acid solution, the molecules of said collagen maintaining the native triple helix structure in the solution at a temperature of +4° C. to +25° C., i.e. retain the ability to form fibrils under physiological conditions. The concentrated solution of the collagen, wherein the collagen content is more than 96% of the dry weight of the total protein, forms stable polymeric structures (hydrogels) under physiological conditions and can be used for the manufacture of biomedical cell based products and three-dimensional tissue-engineered structures without using chemical and/or photochemical cross-linking. These structures can be of heterogeneous density, including structures with a detail resolution of no larger than 0.3 mm, obtained by 3D printing technique, thus allowing incorporating living cells or cell aggregates used in medical practice in the structure. The disclosed collagen solution will also be useful as a material for forming hydrogels used for cell cultures. The invention also provides a process for the preparation of a sterile clear concentrated solution of biocompatible collagen, and uses thereof.

### PRIOR ART

The problem of scarcity of donated transplant organs encourages looking for biomedical technical solutions that do not require the usage of donor grafts. Currently, there is a great number of processes that allow approaching said problem. The essence of the existing processes consists in restoration of the tissue and/or organ integrity and functions using implantable three-dimensional tissue-engineered structures (TES). An alternative may be found in a local insertion of biomedical cell based products (BMCP) consisting of cells and a biopolymer matrix. The scaffolding biopolymer matrix for a localized cell implantation can also contain growth factors that will exert stimulating effects on the cells of the damaged tissue.

The main function of a biopolymer matrix consists in maintaining viability of the cells introduced therein and in a mechanical integration with the surrounding tissues in the place of grafting with subsequent replacement thereof with reconstituted extracellular matrix built by the cells. Thus, the implantable biopolymer matrices should have the following essential properties:
a high biocompatibility of the structure itself;
the absence of immunological reactions to the material constituting said structure; and
a geometrical design of the structure that does not restrict a free liquid flow within the structure.

Considering that the free diffusion rate of metabolites, in particular macromolecules, in hydrogels is low, geometrical dimensions of the matrix surrounding a living cell should not exceed 0.15 mm. It is specifically the porous structure of a biopolymer matrix that allows a culture medium to flow through the structure when the latter is prepared for grafting and provides for the flow of tissue fluid within the grafted structure until a capillary blood supply is formed therein.

Maintaining the cell viability within the biopolymer matrix requires an external supply of nutrients and a removal of the metabolic products; otherwise, cell death in closed ischemic zones cannot be avoided.

3D printing technology is a universal modern technique for producing a biopolymer matrix having a complicated predetermined configuration and a high resolution of details. This technology allows arranging and combining in different ways differentiated living cells and structural elements of scaffolding matrix in space.

Consequently, biopolymer matrices that serve as the basis for forming the structure of BMCPs and three-dimensional TESs should be compatible with both the cells incorporated therein and the living tissues. These matrices should slowly degrade in the organism into harmless components, while being progressively replaced with an extracellular matrix synthesized by the cells themselves. The matrix material and the products of degradation thereof should have no immunogenicity. In order to form a predetermined structure using modern 3D printing processes, a biopolymer matrix should not comprise any fillers or cross-linking agents, which may reduce biocompatibility and increase immunogenicity of the printed article. In other words, the biopolymer matrix should have physical characteristics that would allow producing structures by a 3D printing process with the resolution of details 0.3 mm without using any chemical and/or photochemical cross-linking.

Numerous polymer solutions are disclosed in the literature, said solutions being used for manufacturing BMCPs and three-dimensional TESs by a 3D bioprinting method, including solutions of natural polymer-forming substances like alginate (1), chitosan (2), gelatin (3), hyaluronic acid (4), Matrigel™ (5), type I collagen (6) and solutions of synthetic polymer-forming materials like GelMA (7), Pluronic® F-127 (8). However, only pure collagen meets the requirements to materials for the manufacture of BMCPs and three-dimensional TESs intended for medical applications.

Collagen is the principal structural element of a connective tissue forming the framework of extracellular matrix. Collagen is found in the interstitial tissue of virtually all parenchymatous organs. Collagen is characterized by a significant similarity of the amino acid sequence in various species, which fact allows using animal collagen in clinical practice. At the same time, there are several main challenges in the manufacture of collagen-based medical products, such as immunogenicity of the most of animal collagen preparations caused by a residual contamination with protein and other polymeric substances in the collagen preparations and the presence of endotoxins therein. The manufacture process of sterile collagen preparations without damaging the native structure of the triple helix molecule is also very important. The unsolved problems result in weak mechanical properties of BMCPs and three-dimensional TESs made of the existing collagen preparations. The probability of an immune response to an injected collagen-containing preparation depends on the amount of the collagen form, the amount of the protein and non-protein impurities; therefore, immunogenicity of collagen preparations greatly depends on a degree of purification and the preservation of the native structure of the protein (9, 10). The problem of weak mechanical properties of BMCPs and three-dimensional TESs made of the existing collagen preparations is mainly caused by the fact that the transformation of the solution to the hydrogel state requires a long time (about 30-40 min), and this time is necessary for stabilizing the shape of the printed structure. The structure strengthening is achieved by a chemical modification of the collagen within the produced BMCP and three-dimensional TES, however, a chemical modification of the collagen structure results in the produced article losing its principal property—the biocompatibility (11).

A visco-elastic solution of chemically modified collagen described earlier (12), wherein the concentration of collagen protein is in the range of 5-50 mg/ml, is a technical solution closest to the claimed specific collagen solution from the technical viewpoint. The disadvantage of said preparation consists in a chemical modification of the collagen, which becomes less biocompatible in comparison with the collagen having the structure similar to the native one.

Furthermore, to manufacture BMCPs and three-dimensional TESs, the collagen solution must be sterile and the amount of endotoxins therein must not exceed 10 U/ml, which is not taken into account in the above invention.

Matrigel™ is another close analog of the invention, having the above necessary properties of a collagen preparation (biocompatibility, sterility, endotoxins level).

This collagen preparation was successfully used to manufacture a three-dimensional TES (5). However, is should be noted that the Matrigel-based polymer solution has a major drawback inherent to the matrix nature and the technology for the manufacturing thereof. Matrigel is a collagen-containing preparation of an extracellular matrix protein extract obtained from the EHS sarcoma, a malignant tumor in mice. For that reason, it cannot be used for manufacturing BMCPs and three-dimensional TESs for clinical applications.

Close to the claimed sterile clear concentrated solution of biocompatible collagen is a sterile collagen solution comprising, in addition to the principal protein (collagen) having a concentration in the solution of 12.5 mg/ml, another protein (fibronectin) and a mixture of growth factors. Such complex preparation was successfully used for fertility restoration in model animals (13). The drawback of said preparation consists in its complexity: the presence of fibronectin and poorly characterized mixture of different growth factors, which limits the possible clinical applications thereof. Moreover, the complex composition results in a significant increase of the product cost. It is noted that in the manufacture of BMCPs and three-dimensional TESs, the amount of endotoxins should not exceed 10 U/ml for them to be suitable for a medical application, which is not taken into account in said article.

Another close analog of the invention is a neutralized collagen solution used for manufacturing a membrane for the substitution of a urinary bladder wall (14). Despite the membrane made of this collagen solution was successfully used in in vivo animal models, this solution has a significant drawback consisting in that the used neutralized collagen solution requires a strict adherence to temperature requirements: the temperature should not be lower than 4° C. or higher than 10° C. Non-compliance with said requirements, in particular during a transportation of the solution to the end user, will result in a complete polymerization of the preparation and the impossibility of using thereof as intended, therefore, a commercial use thereof is complicated significantly.

It is noteworthy that the claimed sterile clear concentrated solution of biocompatible collagen is free from the above disadvantage and may be stored at a room temperature for a week. It should be further noted that the claimed solution allows manufacturing clear membranes that remain transparent under physiological conditions. This property will facilitate the work of a surgeon when he/she fixes the membrane at the application site while allowing a complete monitoring of the surgery site.

When it comes to the method of use, the closest analog from the technical viewpoint of the claimed product, which is a sterile clear concentrated solution of biocompatible collagen, is a sterile concentrated neutral collagen solution having a collagen protein concentration in the range of 20 to 40 mg/ml used for manufacturing structures by 3D printing technique (15). Despite the successful use of the collagen preparation for manufacturing three-dimensional structures by 3D-printing, a significant drawback of said preparation consists in the necessity of a strict adherence to temperature requirements, with any non-compliance resulting in impossibility of using the preparation for manufacturing structures by 3D printing. It is noteworthy that the claimed collagen solution is free from said disadvantage, which fact facilitates the logistic significantly and reduces the cost of the product itself. It should be further noted that the claimed solution allows making clear structures. This property allows the users to monitor the behavior of the cells incorporated in the structure both by classic optical microscopy methods and by confocal microscopy methods.

Currently, there are many known processes for producing collagen from different sources of raw material (16, 17, 18, 19, 20, 21). However the processes disclosed in the cited references do not allow producing a sterile collagen solution, wherein the level of endotoxins does not exceed 10 U/ml and wherein the collagen maintains its ability to form solid and clear hydrogels under physiological conditions. Moreover, in order to use such collagen solutions prepared according to the above processes for manufacturing structures with a detail resolution of 0.3 mm by a 3D printing technique, it is necessary to use a chemical modification of the collagen that results in a decrease of biocompatibility of the printed structure.

The closest analog of the invention in what concerns the process for the preparation of a sterile collagen solution and the properties of the prepared collagen solution is the process disclosed in the patent (20). This invention provides a collagen solution having the following properties:
α_{2(I)1}/α_{1(I)1} ratio from 0.48 to 0.52;
sterility in accordance with the European Pharmacopoeia standard;
total nitrogen content of 17.0 to 18.7%;
hydroxyproline content from 12 to 13.9%;
does not comprise tryptophan, aminoglycans and polypeptides having molecular weight of <95000 Da;
lipids <1%;
sulfur-containing ash <2%.

The invention provides a process for the preparation of a collagen solution having said properties, said process comprising two steps:

1) the step of stirring and shearing an acidic collagen solution in a mixer with double transverse cutters, while incrementally increasing the stirring rate by 500-1000 rpm, but not higher than 10,000 rpm, to increase the initial ambient temperature to a controlled maximum temperature not higher than 50° C.; and then

2) the sterilization step in a liquid medium using a membrane filtration or a peracetic acid.

However, it is noteworthy that the disclosed collagen preparation process has a major drawback inherent to the technology for the manufacture thereof. Indeed, the heating of an acidic collagen solution to temperatures above 40° C. may result in a complete denaturation of the collagen, so that it is transformed into a gelatin form with immunogenicity of the produced preparation increasing and physical properties of the solution deteriorating simultaneously, which significantly impairs stability of BMCPs and three-dimensional TESs made from said solution.

Unlike the aforesaid analogs, the claimed sterile clear concentrated solution of biocompatible collagen has no disadvantages mentioned above, as it has the following specific properties resulting from the process for the preparation thereof:
the solution is sterile and maintains biocompatibility and triple helix structure of collagen protein;
the amount of endotoxins in the solution is less than 10 U/ml;
the solution is clear;
the solution is suitable for manufacturing biomedical cell based products;
the solution is suitable for manufacturing three-dimensional tissue-engineered structures with the detail resolution of 0.3 mm;
the solution is suitable for manufacturing transparent structures;
the solution maintains its properties and functionality at a room temperature.

### SUMMARY OF THE INVENTION

The problem solved by the present invention is the provision of a preparation, which is a collagen solution, that is suitable for manufacturing BMCPs and three-dimensional TESs for clinical applications by different processes including 3D printing. It has been hypothesized that in order to improve mechanical characteristics/properties of BMCPs and three-dimensional TESs manufactured by 3D printing with detail resolution of lesser than 0.3 mm without using a chemical modification, concentrated solutions of collagen (concentration higher than 20 mg/ml) would be more suitable than widely used collagen solutions (concentration less than 10 mg/ml). While staying in an acetic acid solution, the collagen proposed for this approach should maintain its triple helix structure and should rapidly (in less than 5 minutes) form stable polymeric structures (hydrogels) when in the physiological conditions.

As there is a need in assessing the efficiency of the use of such collagen preparation from the standpoint of cell behavior within the formed hydrogels, there is a requirement of clarity of such hydrogels, and clear hydrogels may be prepared using clear collagen solutions to start with.

In view of the above, the first aspect of the claimed invention is a collagen solution characterized by the following properties:

### Principal Protein Composition of the Preparation

| | |
|---|---|
| Type I collagen | 96-99% |
| Gelatin (denatured form of type I collagen) | 1-4% |

### Biochemical Characteristics of the Preparation

| | |
|---|---|
| Collagen concentration in the solution | 20-200 mg/ml |
| Solvent | an aqueous solution comprising |
| | 0.0005-0.02M acetic acid |
| Endotoxins | no more than 10 U/ml |
| Sterility | in accordance with the Ph. Eur. |
| | standards. |

Physical Characteristics of the Preparation

Rheological properties: The value of shear modulus of elasticity (G′) exceeds the value of shear modulus of viscosity (G″) by more than 500 kPa.

Transparency: the preparation forms stable transparent polymeric structures (hydrogels) under the physiological conditions, said structures being sufficiently transparent for investigating them by confocal and classic optical microscopy methods; the light transmittance in the wavelength range of 400 nm to 780 nm is no less than 85% with a layer thickness of 1.0 mm and a collagen concentration in the solution of 20 mg/ml.

Polymerization: the polymerization time of the solution, once it is neutralized at a temperature of +37° C., is no more than 5 minutes in the above range of the collagen concentrations in the solution.

The second aspect of the invention is a process for the preparation of a collagen characterized above.

The claimed process for the preparation of collagen comprises several steps to be carried out in a sequential order:

1) extracting a collagen-containing tissue like tendons, placenta of mammals, such as a calf, a pig, a rat or a human, in a solution of diluted (for example, no more than 0.5 M) organic acid after an intense washing out of non-collagen protein impurities with alkaline or neutral aqueous salt solutions (for example with 0.5 M Na₂HPO₄) and of contaminating proteins, glycoproteins, fat-containing aggregates with organic solvents (for example acetone, ethanol, acetonitrile); or extracting a collagen-containing tissue like tendons, placenta of mammals, such as a calf, a pig, a rat or a human, in a solution of diluted (for example, no more than 0.5 M) organic acid after an intense washing out of non-collagen protein impurities with alkaline or neutral aqueous salt solutions (for example with 0.5 M Na₂HPO₄) and of contaminating proteins, glycoproteins, fat-containing aggregates with organic solvents (for example acetone, ethanol, acetonitrile), including treatment of said tissue with proteolytic enzymes cleaving only globular telopeptides (for example, pepsin) (22);

2) repeatedly (for example, no less than 3 times) purifying the acidic aqueous salt extract from potentially immunogenic macromolecules by means of a selective precipitation of the collagen using high concentrations of sodium chloride (such as from 0.7M up to 3.0M) at a low temperature of the solution (below 10° C.) (23);

3) purifying the acidic aqueous salt extract from the remaining impurities of potentially immunogenic macromolecules by adsorption of the remaining impurities on the DEAE-cellulose (23);

4) purifying the extract from pyrogenic impurities by adsorption of lipopolysaccharides on an affinity sorbent (for example, with an immobilized polymyxin (Affi-Prep®) or LPS-specific antibodies) (24);

5) purifying the extract from impurities of common and spore-forming microorganisms and also from supermolecular collagen aggregates by microfiltration of the acidic collagen solution through membranes having a pore diameter of 0.22-0.45 μm (19);

6) bringing the collagen solution to a required concentration of the collagen protein that has molecules maintaining the triple helix structure and a molecular weight of no less than 300 kDa, using one of the known methods: ultrafiltration, evaporation, lyophilization, dilution, or water absorption by insoluble hydrophilic polymers (6, 21, 25).

The proposed process for the preparation of a collagen solution is based on the usage of standard methods of protein purification in a sequential order that were not used before in a combination to produce a new product, in particular a sterile clear concentrated biocompatible viscous aqueous salt acidified solution of collagen, the molecules of said collagen maintaining the triple helix structure, wherein the amount of endotoxins does not exceed 10 U/ml, said product being suitable for use in the preparation of biomedical cell based products and three-dimensional tissue-engineered structures, in particular with a detail resolution of no more than 0.3 mm by 3D printing with possible incorporation of living cells or cell aggregates, in a wide range of medical applications and for cell culturing.

The third aspect of the invention is a process of using the collagen solution characterized above in the manufacture of biomedical cell based products and three-dimensional tissue-engineered structures for following medical applications (but without limitation):
in the replacement surgical repair of urinary bladder defects;
in the development of an artificial analog of cornea;
for introducing embryo cells into a spinal cord in a spinal injury model;
for using a biocompatible carrier for the prolongation of action of growth factors in a model of cryptorchism treatment;
for providing an osteoplastic material in dentistry;
as a biocompatible plate in a brain surgery;
as one of the main constituents of bioinks used in 3D printing of three-dimensional tissue-engineered structures.

### DETAILED DESCRIPTION OF EMBODIMENTS OF THE INVENTION

The claimed sterile clear concentrated solution of biocompatible collagen, the process for the preparation thereof, methods for testing and using the same are further described in more details with reference to examples. The provided examples are for illustrative purposes only and should not be used as a limitation of the scope of the invention.

### Example 1. Process for the Preparation of a Sterile Clear Concentrated Solution of Biocompatible Collagen in a Native Form

Step 1. Extracting

Cleaned swine tendons are successively washed 3 times with a saline solution and 5 times with a distilled water, then the obtained material is ground. Further, collagen is extracted with a 0.5 M acetic acid for 12 to 24 hours at a temperature of +4° C. to +10° C. A non-dissolved tissue is separated from the extract by centrifugation.

Step 2. Differential Reprecipitation

Purification of the acidic aqueous salt collagen extract from potentially immunogenic macromolecules is carried out by a repeated use of a differential collagen precipitation method described in the literature. For this purpose, the concentration of sodium chloride in the aqueous salt collagen extract is increased to 1.0 M. The solution is incubated for 30 minutes at a temperature of +4° C. with constant stirring. The obtained collagen precipitate is separated from the solution by centrifugation. For the precipitate dissolution, it is added with a 0.1 M acetic acid solution based on the following ratio: one gram of the collagen precipitate has to be added with 10 ml of 0.1 M acetic acid. Then the concentration of sodium chloride in the obtained collagen solution is increased to 1.0 M once again and the procedure of collagen precipitation is repeated two more times. The collagen solution obtained from the three precipitations with 1.0 M sodium chloride is neutralized by adding a concentrated solution of NaOH. The concentration of sodium chloride in the obtained neutral aqueous salt collagen solution is increased to 4.0 M, the solution is incubated for 30 minutes at a temperature of 4° C. and with constant stirring. The obtained collagen precipitate is separated from the solution by centrifugation. For dissolution of the precipitate, it is added with a 0.1 M acetic acid solution based on the following ratio: one gram of the collagen precipitate has to be added with 10 ml of the solution containing 1.0 M sodium chloride, 1.0 M urea, 0.05 M Tris-Cl, pH 7.5. Once the precipitate is dissolved, the obtained collagen solution is dialyzed against the solution containing 1.0 M sodium chloride, 1.0 M urea, 0.05 M Tris-Cl, pH 7.5.

Step 3. Removing Remaining Impurities of Potentially Immunogenic Macromolecules

The aqueous salt solution of collagen obtained at step 2 is passed through a chromatographic column with the DEAE-cellulose preliminarily balanced with a water solution containing 1.0 M sodium chloride, 1.0 M urea, 0.05 M Tris-Cl, pH 7.5. The column temperature during the chromatography is maintained at 4° C. and the flow rate is 2.5 column volumes per hour. Under these conditions, the collagen is not retained by the sorbent and is collected in the breakthrough. The obtained collagen solution is further dialyzed against the solution containing 1.0 M sodium chloride, 0.02 M sodium acetate, pH 5.5.

Step 4. Removing Endotoxins

The aqueous salt solution of collagen obtained at step 3 is incubated with a sorbent comprising immobilized polymyxin (Affi-Prep®Polymyxin Matrix) preliminarily washed with an aqueous solution containing 1.0 M sodium chloride and 0.02 M sodium acetate, pH 5.5, for 24 hours at a temperature of +4° C. and gentle stirring. Then the sorbent is separated from the collagen solution by centrifugation. The obtained collagen preparation is dialyzed against an aqueous solution of 0.02 M sodium acetate, pH 5.5 (free of pyrogens).

Step 5. Purification from Impurities of Common and Spore Forming Microorganisms

Purification from impurities of common and spore-forming microorganisms and also from supermolecular collagen aggregates is carried out by a cascade microfiltration through membranes having a pore diameter of 0.45 μm and then 0.22 μm.

Step 6. Bringing the Collagen Solution to a Required Concentration

Bringing the collagen solution to a required concentration of the collagen protein is carried out using one of the known methods: ultrafiltration, evaporation, lyophilization, dilution, or water absorption by insoluble hydrophilic polymers. The method of lyophilization and dilution is considered as an example. The aqueous salt solution of collagen obtained at step 5 is poured in a drying vessel so that the thickness of the layer does not exceed 1 cm and the solution is frozen at −70° C. Then the solution is freeze-dried. Once lyophilized, the dry collagen preparation is weighed and added with a required amount of sterile apyrogenic solution of 0.0005-0.02 M acetic acid. For example, to produce a collagen solution having a concentration of 80 mg/ml, 800 mg of dry collagen is to be added with 9.2 ml of acetic acid solution.

### Example 2. Process for the Preparation of a Sterile Clear Concentrated Solution of Biocompatible Atelocollagen (Collagen Free of Telopeptides)

Step 1. Extracting

Cleaned swine tendons are successively washed 3 times with a saline solution and 5 times with a distilled water, then the obtained material is ground. Further, the collagen is extracted with a 0.5 M acetic acid aqueous solution containing 0.3 g/l pepsin for 12 to 24 hours at a temperature of +4° C. to +10° C. A non-dissolved tissue is separated from the extract by centrifugation.

Step 2. Differential Reprecipitation

Purification of the acidic aqueous salt collagen extract from potentially immunogenic macromolecules is carried out by a repeated use of a differential collagen precipitation method described in the literature. For this purpose, the concentration of sodium chloride in the aqueous salt collagen extract is increased to 1.0 M. The solution is incubated for 30 minutes at a temperature of +4° C. with constant stirring. The obtained collagen precipitate is separated from the solution by centrifugation. For the precipitate dissolution, it is added with 0.1 M acetic acid solution based on the following ratio: one gram of the collagen precipitate has to be added with 10 ml of 0.1 M acetic acid. Then the concentration of sodium chloride in the obtained collagen solution is increased to 1.0 M once again and the procedure of collagen precipitation is repeated two more times. The collagen solution obtained from the three precipitations with 1.0 M sodium chloride is neutralized by adding a concentrated solution of NaOH. The concentration of sodium chloride in the obtained neutral aqueous salt collagen solution is increased to 4.0 M, the solution is incubated for 30 minutes at a temperature of 4° C. and with constant stirring. The obtained collagen precipitate is separated from the solution by centrifugation. For dissolution of the precipitate, it is added with 0.1 M acetic acid solution based on the following ratio: one gram of the collagen precipitation has to be added with 10 ml of the solution containing 1.0 M sodium chloride, 1.0 M urea, 0.05 M Tris-Cl, pH 7.5. Once the precipitate is dissolved, the obtained collagen solution is dialyzed against the solution containing 1.0 M sodium chloride, 1.0 M urea, 0.05 M Tris-Cl, pH 7.5.

Step 3. Removing Remaining Impurities of Potentially Immunogenic Macromolecules

The aqueous salt solution of collagen obtained at step 2 is passed through a chromatographic column with the DEAE-cellulose preliminarily balanced with a water solution containing 1.0 M sodium chloride, 1.0 M urea, 0.05 M Tris-Cl, pH 7.5. The column temperature during the chromatography is maintained at 4° C. and the flow rate is 2.5 column volumes per hour. Under these conditions, the collagen it not retained by the sorbent and is collected from the solution coming out of the column with the sorbent. The obtained collagen solution is further dialyzed against the solution containing 1.0 M sodium chloride, 0.02 M sodium acetate, pH 5.5.

Step 4. Removing Endotoxins

The aqueous salt solution of collagen obtained at step 3 is incubated with a sorbent comprising immobilized polymyxin (Affi-Prep®Polymyxin Matrix) preliminarily washed with an aqueous solution containing 0.02 M sodium acetate at pH 5.5 and 1.0 M sodium chloride for 24 hours at a temperature of +4° C. and gentle stirring. Then the sorbent is separated from the collagen solution by centrifugation. The obtained collagen preparation is dialyzed against an aqueous solution of 0.02 M sodium acetate, pH 5.5 (free of pyrogens).

Step 5. Purification from Impurities of Common and Spore Forming Microorganisms

Purification from impurities of common and spore-forming microorganisms and also from supermolecular collagen aggregates is carried out by a cascade microfiltration through membranes having a pore diameter of 0.45 μm first and then 0.22 μm.

Step 6. Bringing the Collagen Solution to a Required Concentration

Bringing the collagen solution to a required concentration of the collagen protein is carried out using one of the known methods: ultrafiltration, evaporation, lyophilization, dilution, or water absorption by insoluble hydrophilic polymers. The method of lyophilization and dilution is considered as an example. The aqueous salt solution of collagen obtained at step 5 is poured in a drying vessel so that the thickness of the layer does not exceed 1 cm and is frozen at −70° C. Then the solution is freeze-dried. Once lyophilized, the dry collagen preparation is weighed and added with a required amount of sterile apyrogenic solution of 0.0005-0.02 M acetic acid. For example, to produce a collagen solution having a concentration of 80 mg/ml, 800 mg of dry collagen is to be added with 9.2 ml of acetic acid solution.

### Example 3. Relationship Between the Collagen/Gelatin Ratio in a Collagen Solution and the Gelation Time Under Physiological Conditions

Any process used for the preparation of a sterile collagen and storing it for a long time in acetic acid solutions can result in a denaturation of the collagen, in a transition to a gelatin form (an unfolded triple helix of the native structure of the collagen molecule). The presence of gelatin in a native collagen preparation affects the rate of induced gel formation and formation of stable solid structures. In order to make it technically feasible to manufacture biomedical cell based products and three-dimensional tissue-engineered structures using collagen solutions, the transition to a hydrogel state time under physiological conditions should not exceed 5 minutes.

A range of collagen solutions having concentrations of collagen up to 40 mg/ml was prepared according to the technique described in Example 1. Gelatin concentration in the solutions was determined using a commercial ELISA system “Gelatin-test” (000 “Imtek”, Russia). The concentration of gelatin in the solutions did not exceed 1%. In some of the prepared solutions all of the collagen was converted to a gelatin form by heating up to 50° C. for 3 hours. Further, the solutions of collagen having various collagen/gelatin ratios were prepared by mixing the collagen solutions having concentration of collagen of 40 mg/ml with the solutions containing 40 mg/ml of gelatin. The prepared solutions of collagen and gelatin mixture were mixed with a phosphate buffer saline solution at a volume ratio of 1:1 for neutralization. Transition time of the solution into a hydrogel state was measured using a rotary rheometer (Anton-Paar). For this purpose the prepared neutral collagen solutions with different collagen/gelatin ratios were placed between the two rheometer plates with the temperature of the plates being maintained at 37° C. during the whole measurement period. The results provided in the table evidence that when the amount of gelatin in the collagen solution exceeds 4%, the complete solidification time of the gel exceeds 5 minutes.

| | Gelatin | Time of gel |
|---|---|---|
| Collagen | (denatured | formation, |
| solution | type I collagen) | min |
| 99% | 1% | 3.5 ± 0.3 |
| 98% | 2% | 3.9 ± 0.2 |
| 97% | 3% | 4.3 ± 0.1 |
| 96% | 4% | 4.8 ± 0.3 |
| 95% | 5% | 5.3 ± 0.3 |

### Example 4. Effect of Collagen Concentration on the Detail Resolution in 3D Printing

In order for the cells placed in a three-dimensional tissue-engineered structure to survive, it is necessary to ensure an efficient elimination of metabolic by-products of the cells and a supply of nutrients to the cells. Therefore, the matrix surrounding a cell should not exceed 0.3 mm in size. A series of collagen solutions having different collagen concentrations was prepared in accordance with the technique described in Example 1. The collagen solutions were mixed with a phosphate buffer saline solution at a volume ratio of 1:1 for neutralization, and then placed in a 3D printer syringe. The syringe was equipped with a conic nozzle having the outlet diameter of 0.15 mm. Printing was carried out in a commercial extrusion 3D printer (RegenHU), which ensured collagen cooling in the syringe during printing and heating of a surface on which the printing was performed. The detail resolution in the printed structure (lattice) was determined by optical microscopy methods. The results provided in the table evidence that the detail resolution is maintained at a required level when a concentration of collagen in the neutral collagen solution is more than 20 mg/ml. However, when the concentration exceeds 200 mg/ml, there were difficulties as it was impossible to push out the collagen having such concentration from the nozzle of the diameter used in the experiment. Thus, the optimal range of collagen concentrations for 3D printing is the range from 20 mg/ml to 200 mg/ml.

| Collagen | Detail resolution of |
|---|---|
| concentration, | a printed structure, |
| mg/ml | mm |
| 10 | 0.8 ± 0.1 |
| 15 | 0.6 ± 0.2 |
| 20 | 0.3 ± 0.1 |
| 40 | 0.3 ± 0.1 |
| 60 | 0.27 ± 0.05 |
| 100 | 0.2 ± 0.1 |
| 130 | 0.17 ± 0.03 |
| 170 | 0.17 ± 0.02 |
| 200 | 0.17 ± 0.02 |
| 210 | — |
| 240 | — |

### Example 5. Effect of the Buffer in the Collagen Solution on the Gelatin Amount in Said Solution During a Long-Term Storage

As shown in example 3, gelatin in the collagen solution has a significant effect on the time of transition of the collagen into a gel form. It is known that collagen is transformed into a gelatin form during a long-term storage in acidic solutions. From the point of view of an end user, the product which is a solution of the preparation of purified collagen with the molecules of the collagen maintaining a triple helix structure under the given conditions, should have a shelf life of at least one year. A range of collagen aqueous solutions having different concentrations of acetic acid was prepared in accordance with the technique described in Example 1. These preparations were stored for 1 year at a temperature of 4-10° C. The amount of denatured collagen therein was measured using a commercial ELISA test system “Gelatin-test” (000 “Imtek”, Russia). The results provided in the table evidence that the optimal range of acetic acid concentration in an aqueous solution in terms of the collagen stability is from 0.0005 M to 0.02 M. It should be noted that it was impossible to completely dissolve collagen in an acetic acid solution having a concentration below 0.0005 M.

| | Amount of gelatin in |
|---|---|
| Concentration of | the collagen solution |
| acetic acid, | after one year, |
| M | % |
| 0.0005 | 1.1 ± 0.2 |
| 0.001 | 1.2 ± 0.3 |
| 0.015 | 2.0 ± 0.1 |
| 0.02 | 2.5 ± 0.3 |
| 0.03 | 4.2 ± 0.3 |
| 1.0 | 20 ± 2 |

### Example 6. Effect of Rheological Properties of a Collagen Solution on 3D Printing of Multilayer Macroscopic Objects

In order to manufacture large three-dimensional tissue-engineered structures by a 3D printing process, the collagen solution that is pushed out through a fine needle should maintain its shape for 5 minutes until it is completely solidified due to the gel formation under physiological conditions. This is only possible with collagen solutions having values of shear modulus of elasticity (G′) exceeding the value of the modulus of viscosity (G″). Therefore, the difference between these two values has to be determined. A range of collagen solutions having concentrations of collagen of 80 mg/ml in a 0.02 M acetic acid solution was prepared according to the technique described in Example 2. The solvent was added with urea to influence the values of G′ and G″ moduli without changing the collagen concentration. Several groups of collagen solutions having different G′ and G″ modulus values were prepared in such a manner, 10 samples in each group. The modulus values were measured using a rotary rheometer (Anton-Paar). After that, the prepared collagen solutions were tested for suitability for a direct extrusion 3D printing of multilayer macroscopic objects: the ability to maintain the shape for 5 minutes. The results of the test are provided in the table.

| The difference between G′ and G″, | |
|---|---|
| kPa | Suitability for 3D printing |
| 100 ± 10 | No |
| 300 ± 14 | No |
| 450 ± 25 | No |
| 500 ± 7 | Yes |
| 1000 ± 40 | Yes |
| 3000 ± 80 | Yes |

### Example 7. Use of a Sterile Clear Concentrated Solution of Biocompatible Collagen for the Preparation of a Biopolymer Matrix with Human Living Cells Incorporated Therein

The collagen prepared in example 1 in an aqueous solution of 0.001 M acetic acid with the collagen concentration of 40 mg/ml is used here. The product is mixed with a solution containing a suspension of living cells at a volume ratio of 1:3 or 1:1. Once introduced into a human organism, the produced biomedical cell based product forms a matrix that maintains functional activity of the human cells contained therein. For example, by mixing the collagen with a suspension of human mesenchymal stem cells isolated from umbilical cord blood it is possible to manufacture a biomedical cell based product for restoring the limb motor function lost following a spinal cord injury.

### Example 8. Use of a Sterile Clear Concentrated Solution of Biocompatible Collagen for the Preparation of a Strong Membrane Suitable for Implantation with the Purpose of Surgical Replacement of Damaged Natural Biomembranes

The collagen having a concentration of 25 mg/ml in an aqueous solution of 0.01 M acetic acid was prepared in accordance with the technique described in Example 2. 0.5 ml of said collagen was placed in a well of a 24-well polystyrene plate (92424, Tissue Plastic Production). The plate with the protein solution was incubated at a temperature of +37° C. in ammonia vapor for 3 hours to achieve a smooth layer. The incubation resulted in a formation of a collagen hydrogel in each well of the plate. After that, the produced hydrogels were removed from the plate and dried under sterile conditions at a temperature not higher than +30° C. and a relative humidity of 40%. The drying was considered as completed when the collagen hydrogel was transformed into a rigid glass-like membrane. The membrane produced in this way can be used for a surgical replacement of damaged natural biomembranes, for example, in a urinary bladder wall closure, for repairing dura and cornea.

### Example 9. Use of a Sterile Clear Concentrated Solution of Biocompatible Collagen for the Preparation of a Clear Hydrogel Supporting Cell Proliferation in a Three-Dimensional Culture

The collagen having a concentration of 40 mg/ml in an aqueous solution of 0.01 M acetic acid was prepared in accordance with the technique described in Example 2. 1.0 ml of the collagen solution was added with 3.0 ml of a mammalian cell suspension (for example, human fibroblasts) in a culture medium and quickly mixed at a temperature not higher than +10° C. The obtained mixture was carefully placed in wells of articles for cell culture (for example, using a 24-well plate), so that the thickness of the produced hydrogel layer did not exceed 1 mm, and was incubated at 37° C. for 30 minutes. The collagen hydrogel produced in this way had a transparency sufficient for an investigation by confocal or classic optical microscopy methods for the whole cell culture period. Moreover, the produced hydrogel ensures cell proliferation and maintains cell viability.

### CITED LITERATURE

1. Markstedt K, Mantas A, Tournier I, et al. 2015. 3D Bioprinting Human Chondrocytes with Nanocellulose-Alginate Bioink for Cartilage Tissue Engineering Applications. Biomacromolecules 16: 1489-1496.
2. Zhang Y, Yu Y, Ozbolat I T, 2013. Direct Bioprinting of Vessel-Like Tubular Microfluidic Channels. J. Nanotechnol. Eng. Med. 4: 20902.
3. Wang X, Yan Y, Pan Y, Xiong Z, et al. 2006. Generation of three-dimensional hepatocyte/gelatin structures with rapid prototyping system. Tissue Eng. 12: 83-90.
4. Skardal A, Zhang J, Prestwich G D, 2010c. Bioprinting vessel-like constructs using hyaluronan hydrogels crosslinked with tetrahedral polyethylene glycol tetracrylates. Biomaterials 31: 6173-6181.
5. Snyder J E, Hamid Q, Wang C, et al. 2011. Bioprinting cell-laden matrigel for radioprotection study of liver by pro-drug conversion in a dual-tissue microfluidic chip. Biofabrication 3: 34112.
6. Rhee S, Puetzer J L, Mason B N, et al. 2016. 3D Bioprinting of Spatially Heterogeneous Collagen Constructs for Cartilage Tissue Engineering. ACS Biomater Sci Eng; 2(10): 1800-1805.
7. Du M, Chen B, Meng et al. 2015. 3D bioprinting of BMSC-laden methacrylamide gelatin scaffolds with CBD-BMP2-collagen microfibers. Biofabrication 7: 44104.
8. Wu W. DeConinck A, Lewis J, 2011. Omnidirectional printing of 3D microvascular networks. Adv. Mater. 23: H178-83.
9. Lynn A K, Yannas I V, Bonfield W. 2004. Antigenicity and Immunogenicity of Collagen. J Biomed Mater Res. 71B(2): 343-354
10. Timpl R. 1982. Antibodies to collagen and procollagen. Meth. Enzymol. 82: 482-98.
11. Courtman D W, Errett B F, Wilson G J. 2001. The role of crosslinking in modification of the immune response elicted against xenogenic vascular acellular matrices. J Biomed Mater Res; 55(4): 576-586.
12. U.S. Pat. No. 4,713,446A.
13. Kamalov A A, Kirpatovsky V I, Ohobotov D A, et al. 2017. The application of a novel biomaterial based on the secreted products of human mesenchymal stem cells and collagen for spermatogenesis restoration in the model of experimental cryptorchidism. Res J Pharm Biol Chem Sci; 8(1):2083-2094.
14. Kirpatovckii V I, Kamalov D M, Efimenko A Y, et al. 2016. Urinary bladder substitution using combined membrane based on secretions of human mesenchymal stem cells and type I collagen. Urologiia; (6):34-42.
15. Osidak E O, Karalkin P A, Osidak M S, et al. 2019. Viscoll collagen solution as a novel bioink for direct 3D bioprinting. J Mater Sci: Mater Med. In press.
16. U.S. Pat. No. 4,389,487A
17. RU 2272808
18. RU 2094999
19. RU 2214827
20. RU2188206C2
21. US20170334969A1
22. KyxapeBa [symbol] B, [symbol] [symbol] , [symbol] EB. 2010. MeTO [symbol] [symbol] [symbol] [symbol] [symbol] [symbol] [symbol] [symbol] [symbol] . [symbol] . 52(7): 597-602. (Kukhareva L. V., Shamolina I. I., Polevaya E. V. 2010. Method for producing collagen from a calf skin for tissue engineering and cell culturing. Tsitologya. 52(7): 597-602—in Russian language)
23. Deyl Z, Miksik I, Eckhardt A. 2003. Preparative procedures and purity of collagen proteins. J Chromatography. 790: 245-375.
24. Hirayama C. Sakata M. 2002. Chromatographic removal of endotoxin from protein solutions by polymer particles. J Chromatography. 781(1-2): 419-432.
25. Tidu A, Ghoubay-Banallaoua D, Lynch B. 2015. Development of human corneal epithelium on organized fibrillated transparent collagen matrices synthesized at high concentration. Acta Biomaterialia. 22(2015): 50-58.

1. A sterile clear viscous aqueous salt solution containing 0.1 mN-20 mM of an acid maintaining acidity of the solution in the pH range of pH 2.5 to pH 3.5 and a purified collagen that amounts to no less than 96% of the total protein by dry weight, said solution comprising no more than 4% of the denatured collagen by dry weight at a temperature of +4° C. to +25° C., and said collagen retaining the ability to form fibrils and stable hydrogels under physiological conditions.

2. The solution according to claim 1, wherein the purified collagen comprises type I collagen.

3. The solution according to claim 1, wherein the collagen is an animal collagen.

4. The solution according to claim 1, wherein the collagen is a human collagen.

5. The solution according to claim 1, wherein the collagen concentration in the solution is in the range from 20 mg/ml to 200 mg/ml.

6. The solution according to claim 1, wherein acetic acid or other organic acid is used as the acid maintaining the acidity value in the pH range of pH 2.5 to pH 3.5 at the above concentration thereof in the solution.

7. The solution according to claim 1, wherein the value of shear modulus of elasticity (G′) exceeds the value of modulus of viscosity (G″) by more than 500 kPa.

8. The solution according to claim 1, wherein sterility of the solution complies with the European Pharmacopoeia sterility standard.

9. The solution according to claim 1, wherein the amount of endotoxins does not exceed 10 U/ml.

10. The solution according to claim 1, wherein the solution forms stable transparent polymeric structures (hydrogels) under physiological conditions, said structures being sufficiently transparent for investigating them by confocal and classic optical microscopy methods; the light transmittance in the wavelength range of 400 nm to 780 nm is no less than 85% with a layer thickness of 1.0 mm and a collagen concentration in the solution of 20 mg/ml.

11. The solution according to claim 1, wherein the polymerization time of the solution, after it is neutralized at a temperature of +37° C., is no more than 5 minutes in the above range of the collagen concentrations in the solution.

12. A process for the preparation of a collagen solution according to claim 1, comprising the following steps to be carried out in a sequential order:
1) extracting a collagen-containing tissue in a solution of diluted (for example, no more than 0.5 M) organic acid after an intense washing out of non-collagen protein impurities with alkaline or neutral aqueous salt solutions and of contaminating proteins, glycoproteins, fat-containing aggregates with organic solvents;
2) repeatedly purifying the acidic aqueous salt extract from potentially immunogenic macromolecules by means of a selective precipitation of the collagen with high concentrations of sodium chloride at a low temperature of the solution (such as from +4° C. to ±10° C.);
3) purifying the acidic aqueous salt extract from the remaining impurities of potentially immunogenic macromolecules by adsorption of the remaining impurities on the DEAE-cellulose;
4) purifying the extract from pyrogenic impurities by adsorption of lipopolysaccharides on an affinity sorbent, for example, with an immobilized polymyxin or LPS-specific antibodies;
5) purifying the extract from impurities of common and spore-forming microorganisms and also from supermolecular collagen aggregates by microfiltration of the acidic collagen solution through membranes having a pore diameter of 0.22 μm-0.45 μm;
6) bringing the collagen solution to a required concentration of the collagen protein that has molecules having a triple helix structure and a molecular weight of no less than 300 kDa, using a method like ultrafiltration, evaporation, lyophilization, dilution, or water absorption by insoluble hydrophilic polymers.

13. A process according to claim 1,
wherein said extraction step includes treatment of said tissue with proteolytic enzymes cleaving only globular telopeptides (for example, pepsin.

14. A method for preparation of a biopolymer matrix with human living cells incorporated therein wherein a collagen solution according to claim 1, is admixed with a suspension of human living cells to form a biopolymer matrix with human living cells incorporated therein with the purpose to manufacture a biomedical cell based product or a three-dimensional tissue-engineered structure for a medical application.

15. A method of producing a bioink which comprises incorporating a collagens solution according to claim 1, into a bioink configured to be used in 3D printing with a detail resolution of lower than 0.3 mm or in bioprinting, complex structures with living cells incorporated therein.

16. A method of preparing a stable transparent polymeric structures which comprises forming a hydrogel from a collagen solution according to claims 1, 11 wherein said hydrogel is configured for maintaining cell growth and differentiation in a three-dimensional culture, wherein transparency is a condition for performing investigations by confocal and classic optical microscopy methods.

17. A method of preparing a stable transparent polymeric structures which comprises forming a hydrogel from a collagen solution according to claim 1, said hydrogel being configured for use as a material for manufacturing clear collagen membranes suitable for implantation with the purpose of surgical replacement of a damaged natural biological membrane, in cornea, urinary bladder wall, tympanic membrane, dura, etc.

18. The process according to claim 12, wherein said collagen-containing tissue is selected from tendons or placenta of a mammal, selected from the group consisting of calves, pigs, rats and humans.

19. The process according to claim 12, wherein said repeated purification of the acidic aqueous salt extract from potentially immunogenic macromolecules comprises effecting selective precipitation of the collagen at least three times with high concentrations of sodium chloride of from 0.7M up to 3.0M at a temperature of the solution of from +4° C. to +10° C.
